(19) United States
(12) Patent Application Publication
(10) Pub. No.: US 2002/0002198 A1
(43) Pub. Date: Jan. 3, 2002

(54) Chemical synergy to elevate growth hormone release in vertebrates

(76) Inventor: Tyler B. Parr, San Diego, CA (US)

Correspondence Address: Tyler Parr, Ph.D., P.O. Box 371, Chula Vista, CA, 91912, US

(21) Appl. No.: 09/835,501

(22) Filed: Apr. 16, 2001

Related U.S. Application Data
(60) Provisional application No. 60/197,470, filed on Apr. 17, 2000.

(51) Int. Cl. A61K 31/4172; A61K 31/22

(52) U.S. Cl. 514/400; 514/550

(57) ABSTRACT

Growth hormone (GH) release in vertebrates may be augmented by an oral dietary supplement composed of acetyl-l-carnitine and l-ornithine acting in synergy. Augmentation is most efficacious by ingestion at night sleep after a short fast, but may be used during the day. Human dosages in subgram levels allow precise and reliable control of the level of augmented GH release over greater than one order of magnitude range above normal levels. This method can: return aging declined GH release to young adult levels, cause rapid fat loss without protein loss or extreme hunger, enable prolonged wakeful alertness and strength during emergencies, promote anabolic function in catabolic disease or trauma, and rapidly mature domestic animals.

### CROSS-REFERENCE TO RELATED APPLICATION

PPA JC553USPTO No. 60/197,470 Apr. 17, 2000

### STATEMENT REGARDING FEDERALLY SPONSORED RESEARCH

(NOT APPLICABLE)

### BACKGROUND

1. Field of Invention

This invention relates to endocrinology, as a method to augment normal growth hormone release in humans and other vertebrates.

2. Description of Prior Art

Growth Hormone Control and Release

Growth hormone (hereafter abbreviated as ‘GH’) exercises the highest hormonal control level of anabolism (biological synthetic processes) in the vertebrates. GH has a major influence on growth rates and also on maintenance of healthy tissue. GH is released from the pituitary gland by a secretion process that we will refer to as GH release. GH arises in evolution with the vertebrate group, all vertebrates are subject to the technique described in this patent application. Gradual decline in the GH release during aging and the more rapid declines in pathological conditions are detrimental to health maintenance. Rates of growth maturation of domestic animals are also increased by augmented GH release levels. Elevation of GH release has a downstream consequence of elevating insulin like growth factor 1 (IGF-1). Elevation of IGF-1 leads to elevated protein synthesis and other anabolic activities. For these reasons, a wide variety of techniques have been employed to increase the level of GH in vertebrates. These various techniques can be divided into two general categories: 1.) delivery of exogenous GH (not made by internal body), and 2.) drugs, chemicals or regimes to augment endogenous (one's own) GH release.

Exogenous Growth Hormone Delivery

Human GH produced by recombinant DNA technology is described in U.S. Pat. No. 5,855,920. Recombinant created GH has been delivered by injection and topical mucus membrane absorption techniques. The topical mucus membrane absorption has been described in PCT Pat. No. WO9959543-A. An untoward consequence of delivery of exogenous (external sourced) GH is the feedback suppression of endogenous production. A control region of the brain called the hypothalamus senses circulating GH and the down stream levels IGF-1. Sensed levels cause feedback modulation of new production of GH. The hypothalamus acts to maintain a age specific set levels of GH. Both the number of GH producing cells and the amount of endogenous GH release are declined by this feedback. Thus, GH augmentation by exogenous delivery does nothing to solve the underlying problem at the level of the hypothalamus. This is recognized to be a fundamentally flawed way to intervene in complicated hormonal feedback systems. A corresponding example is the feedback shrinking of testicular tissue in athletes who seek muscle bulk by injection of exogenous androgens. Exogenous hormone delivery is used when absolutely critical, as in the case of insulin. It is rarely simple or without untoward consequences.

Agents that Augment Endogenous GH Release

Drugs or chemicals that augment endogenous GH release can be subdivided into those that: directly stimulate the release of GH from the pituitary gland, and primarily hypothalamic control augmentation of GH release.

Many drugs employed as GH releasers (called GH secretagogues) circumvent the normal hypothalamic control system to cause GH release by acting directly on the pituitary. Most of these do so by binding receptors other than the normal GHRH receptor. An example of the oral drug based secretagogue is the oligopeptide labeled GRP-6 covered in U.S. Pat. No. 4,411,890. This evasion of normal hormonal control systems for GH release has problems similar to the exogenous GH addition. Most of these GH secretagogues do not act through the normal hypothalamic control mechanism. Normally, hypothalamic release of a hormone called ‘growth hormone releasing hormone’ (GHRH) triggers subsequent pituitary release of GH. The hypothalamic release of GHRH stimulus is also needed for maintenance of sufficient number of GH producing cells and levels of GH stored in those cells. Bypassing this system will gradually deplete both number and content of GH producing pituitary cells. Thus, this resembles the delivery of exogenous GH, with a similar short sighted intervention.

Another class of GH secretagogues that actually bind to the pituitary GHRH receptors are called GHRH mimics. These GHRH mimic secretagogues are drugs that also bypass hypothalamic controls by impersonating the activity of genuine GHRH. Almost all of these drugs are modified forms of the natural hormone GHRH that are injected to act downstream of the hypothalamus to trigger GHRH receptor caused GH release from the pituitary. GH mimics do not augment proper hypothalamic controls over GH. As such, they do not correct the long term aging problems involving a decline in the hypothalamic function. In this case, the normal hypothalamic based GHRH release is feedback inhibited by mimic generated GH production. Again, this is intervention in a complex hormonal feedback system that slights the feedback dynamics of the whole system.

A naturally occurring peptide that promotes immediate GH release has been discovered. This protein, called ghrelin, has been reported to trigger immediate pituitary GH release by binding to a non-GHRH receptor (1). This protein is not believed to play a large role in total 24 hour GH release. Some 80-90% of GH release in males and post menopausal females occurs via hypothalamic control of night time GH release within the first 3 hours of sleep (2,3). Premenopausal women have comparable night time and day time GH release. Day time releases are due to estrogen spike triggered hypothalamic controlled GH release (4). Ghrelin may play only a small part in total GH release.

Hypothalamic Acting GH Releasers

Hypothalamic control of GH release is a dynamic between inhibitory somatostatin levels and the pro-release GHRH secretion. Complex networks of different neuron types also influence this somatostatin versus GHRH opposition. Various drug antagonist or agonists of different neuron types can affect this outcome (5). Our concern here is with relatively simple widely available chemical modulators of this system. Chemicals that directly augment hypothalamic triggered GH release can be further divided into: somatostatin inhibitors, GHRH release enhancers, and unknown action hypothalamic affecters.

Simple chemicals acting as somatostatin inhibitors include a wide variety of L-amino acids. The most potent of these amino acids are ornithine, arginine, lysine, and histidine. The long established ‘arginine provocative test for GH competence’ involves an intravenous administration of 0.5 grams of L-arginine per kilogram of body mass. This represents 30 grams per a 60 kilogram human (6,7). Oral ingestion of such large doses of single basic amino acids induces nausea and other digestive disorders. The lowest oral dose of the amino acid l-arginine that augmented normal night time GH release is greater than 3 grams (8). These doses are not well tolerated, causing digestive distress. The amino acid L-ornithine alone has been patented as a immune system enhancer in U.S. Pat. No. 5,576,351, but not for direct use as GH enhancement. Another possible somatostatin inhibitor that augments GH release is 2-acylaminopropanamides covered in patent PCT WO 97/06803, but these must be administered with the hormone GHRH.

Simple non-drug chemicals that act as GHRH production enhancers (alone) are not known, but many drug compounds influence GHRH production and release.

Some simple chemicals that act at the hypothalamic level to augment GH release have not been characterized as to the exact mode of action. Gamma-hydroxybutyrate administered at multi-gram levels at night can double normal GH release. PCT Pat. No. WO9640105-A describes this process. It is not known how gamma-hydroxybutyrate accomplishes this augmentation of GH release. High levels of gamma-hydroxybutyrate lead to extremely deep sleep that has been linked to dangerous coma like sleep with reported pathology (9). Since 1990, gamma-hydroxybutyrate has been listed as a banned substance by the FDA. Another GH secretagogue, described in PCT Pat. No. WO9744042-A, is also reported to improve sleep. Since sleep satisfaction and GHRH release are known to be associated, this drug is likely to affect hypothalamic function. This GH secretagogue is a pharmaceutical drug requiring a doctors' presciption.

Intellectual Creation of This New Technique of Augmented GH Release

This method was conceived as a consequence of a new theory of aging formulated by Dr. Parr. This ‘Hormonal Imbalance-Growth Factor Exposure’ theory of aging is detailed in currently published articles (10, 11, 12, 13). This theory of aging states that declined maximal potential mitochondrial energy production with age is the principal cause of gradual maintenance declines seen in aging. These mitochondrial changes are a consequence of declined growth hormone support for fat metabolism that elevates the daily exposure to the naturally occurring compound acetyl-l-carnitine. Pharmacological elevated levels of acetyl-l-carnitine temporarily restores youthful maximal mitochondrial energy production capacity to old animals to that of a young adult animal. Underlying this temporary restoration is the return to a youthful level of the absolutely required inner mitochondrial membrane support phospholipid cardiolipin. Cardiolipin levels per unit of mitochondrial protein are known to decline with age and youthful levels of cardiolipin are required for maximal mitochondrial energy production capabilities.

Pharmacological levels of intravenously administered acetyl-l-carnitine restore mitochondrial maximal energy production to youthful levels in aged animals within an hour or two (14). This is a temporary consequence of the elevated acetyl-l-carnitine level, but lasts only for a short duration of a few hours. The level of mitochondrial maximal energy production controls the relative cellular allocation of energy to various subcellular processes like ion pumping, protein synthesis, and other processes like hormone release (15). Thus, a lower maximal mitochondrial energy production (state 3 level) would slight protein synthesis relative to the more survival critical ion pumping. Over the long term, this results in the impaired investment in protein synthetic upkeep of youthful tissue health and function, which is precisely what is observed in the long gradual aging process.

Acetyl-l-carnitine given to aging animals or humans had beneficial effects on various brain neurotransmitters and receptors (16). The combination of orally administered acetyl-l-carnitine and other naturally occurring growth hormone release stimulants like l-arginine or l-ornithine (17) might improve age declined hypothalamus/pituitary function and restore a more youthful growth hormone output. Oral ingestion of the l-arginine alone, in doses of up to 6 gram at night, does not elevate growth hormone secretion (8, 18).

Adult nightly growth hormone secretion occurs primarily in the first 1.5 to 3 hours of night time sleep, and thus, this period was the particular target time for taking this mixture. Fasting for 4 hours should decrease competition for absorption of the mixture in the upper gastrointestinal tract.

The discovery of a remarkable and extremely powerful synergy between oral ingestion of very small amounts of acetyl-l-carnitine and l-ornithine to augment normal GH release is the basis of this patent. The at sleep ingestion of these compounds results in a GH surge at 1.5 hours and 3 hours into sleep. This is fully consistent with a hypothalamic control mechanism where GH is released during slow wave sleep at the end of the 1.5 hour sleep cycle (4). It is inconsistent with a ghrelin type of immediate GH release.

Increasing GH release will reinforce the anti-aging process for the whole body, as it leads to increases in the whole body levels of acetyl-l-carnitine through relatively increased fat metabolism. This creates an ‘all tissue’ improvement in mitochondrial cardiolipin levels and thus such energy dependent functions as increased protein synthesis. This represents a ‘whole body’ feedback system that appears to mutually regulate GH release and subsequent maintenance levels by energy and protein synthesis.

Specific Background Information and Patents Related to Acylcarnitines and Ornithine, etc.

Acetyl-l-carnitine has been found to synergize with a number of other substances. Acetyl-l-carnitine and reservatrol have been reported to synergize in protection against various cerebrovascular involving pathologies like Alzheimer's disease and various other age related dementia as reported in PCT Pat. No. WO0021526-A. An antioxidant combination of acetyl-l-carnitine and alpha-lipoic acid has been reported to offer synergy in neuroprotection in diabetes in PCT Pat. No. WO0011968-A Another acylcarnitine derivative, propionyl l-carnitine, is reported to act in synergy and a flavonoid and other compounds to provide protection against thrombosis and atherosclerosis in PCT Pat. No. WO0028986-A.

Various acylcarnitines alone or in combinations have been patented for treatment of various diseases in U.S. Pat. No. 6,166,077. Combinations of l-carnitine and the l-carnitine metabolic precursor gamma-butyrobetaine have covered in U.S. Pat. No. 5,240,961 for the purpose of elevating serum levels of insulin-like growth factor 1 and osteocalcin levels.

No patent reports have been discovered by the author to indicate any prior report of a specific synergy between only acetyl-l-carnitine and l-ornithine or any of the other acceptable substitutes for this technique of elevating GH release. Acetyl-l-carnitine and numerous amino acids (including ornithine) and other components together have been claimed in U.S. Pat. No. 5,817,329 to support increase in muscle mass of athletes without any claim of augmenting GH release. Acetyl-l-carnitine and other acylcarnitines with or without other hormones have been reported to augment insulin like growth factor 1 (IFG-1) levels in a variety of pathology including acquired immune deficiency (HIV) in PCT Pat. No. WO9801128. This patent involve a combination of various acylcarnitines along with GH administration as well as other bioactive compounds, but does not suggest or establish a specific synergy requiring only low levels of acetyl-l-carnitine and l-ornithine alone.

An enteral or parentral dietary supplement composed of L-ornithine or l-arginine and multiple other amino acids for stimulation of an impaired immune system is covered by U.S. Pat. No. 5,576,351. This regime suggest some 15 to 35 grams per day of arginine and ornithine combined. This mixture is administered through surgery implanted tubing into the gut of immune compromised individuals. Thus, there is no comparison to oral administration of minimal amounts of acetyl-l-carnitine and l-ornithine that act by a specific synergy to augment GH release. A non-steroidal anabolic nutrient supplement mixture of minerals and amino acids including ornithine is claimed to increase muscle mass in PCT Pat. No. WO 98/44793. A nutritional supplement consisting of branched chain amino acids and another group of amino acids including omithine has been claimed to restore GH levels in PCT Pat. No. WO 00/64283. This last formulation does not include acetyl-l-carnitine.

### SUMMARY

Growth hormone (GH) release in vertebrates may be augmented by ingestion of an oral dietary supplement comprised of very small amounts of acetyl-l-carnitine and l-ornithine acting in synergy. This augmentation is most efficacious at night, but can also be used under specific conditions during the day. The magnitude of GH augmentation is precisely controlled by the amounts and proportions of the two chemicals. With his method, aging declined growth hormone release can be elevated and maintained at a young adult level. Greater augmentation can be used to rapidly reduce body fat, rapidly grow immature domestic animals, and treat catabolic medical conditions. This technique appears to enhance hypothalamic function in general, and may augment a variety of other physiological processes.

Object and Advantages

Accordingly, several objectives and advantages of my invention are:

1.)to augment GH release in vertebrates by an oral dietary supplement ingestion of very small amounts of two widely available and inexpensive chemicals acting in synergy;

2.) to precisely and reliably control levels of augmented GH release by small changes in dosage of these chemicals without any attenuation of action over time;

3.) to employ specific temporal scheduling of dietary supplement ingestion under a specified fasting condition to maximize augmentation of GH release;

4.) to augmenting GH release by augmenting normal hypothalamic processes without triggering feedback complications;

5.) to augment a broader hypothalamic enhancement that also increases other hypothalamic driven hormonal release processes like male androgen production; and

6.) to cause a temporary reconditioning of hypothalamic function in aging vertebrates to a more youthful functional status with a wide variety of consequent physiological benefits.

This remarkable synergy at very low levels of two normally occurring natural components of vertebrate bodies, suggests a more profound underlying control system. Temporally targeted supplementation with very low levels of these normal endogenous compounds may be productively intervening on a not yet recognized ‘whole body feedback system’ of enormous physiological consequences. The author of this patent application has already suggested in scientific publication that the gradual decline in this underlying ‘whole body feedback’ may be largely responsible for the youthful to late midlife gradual aging process (13).

### DRAWING FIGURES

NOT APPLICABLE

### DESCRIPTION—PREFERRED EMBODIMENT AND OPERATION

An intake of two substances acting by synergy, leads to a controllable elevation of night time growth hormone release. These two substances will be called a component 1 and a component 2. This magnitude of this synergy is controlled by a standard level of said component 1 and increased levels of said component 2. These substances can be administered by any suitable physiological technique. The components can be administered as a single mixture or as separate intakes. Either or both the components may be taken as liquid or solid formulations. Both the components must be taken within an hour of night time sleep and 3 to 4 hours after the last meal. It is critically important to understand that this specific synergy is abolished by intake of any other amino acids that act as competition for biological uptake. No intake of other amino acids are permitted in the 3 to 4 hours from the last meal to the intake of the component 1 and the component 2. This distinction clearly separates this method from all others employing multiple amino acids that are not exclusively from the component 1 and the component 2.

The component 1 may be any acetyl-l-carnitine or any acylated l-carnitine with a acyl group of 6 carbons or less. The component 1 may also be any mixture of the component 1 substances. The component 1 may be formulated as any pharmacological acceptable salt. The component 1 can be administered in a range of 10 milligrams to 20 grams depending upon the physiological effect desired and animal mass. The preferred choice for the component 1 is acetyl-l-carnitine alone.

The component 2 may be any substance of the following: l-ornithine, l-arginine, l-lysine, l-histidine, l-phenylalanine, l-leucine, l-valine, l-methionine, and l-threonine. The component 2 may also be any mixture of the named substances. The component 2 may be formulated as any pharmacological acceptable salt. The component 2 can be administered in a range of 1 milligram to 10 grams depending on the physiological effect desired and the animal mass. The preferred choice for the component 2 is l-ornithine alone.

The preferred method of administration is oral ingestion of a combined mixture of the component 1 and the component 2 as a dietary supplement in a fast dissolving gelatin capsule. Oral ingestion must take place within an hour of night time sleep. Three to four hours must have elapsed from the last meal before oral ingestion.

Specific Applications and Physiological Doses

1. Elevation of Growth Hormone Release in Aging Humans

An orally ingested mixture of 500 milligrams of acetyl-l-carnitine (the component 1) and 20 to 40 milligrams of L-ornithine (the component 2) is taken just before night sleep. This procedure may be undertaken at any adult age, but is best chosen after 30 years of age. This process can be continued indefinitely. Adjustment of the component 2 dosage will correspond to individual circumstances. This same technique may be used by body builders to increase muscle mass and reduce fat levels.

2.) Rapid Fat Loss by Short Term Elevation of Nightly Growth Hormone Release

Short term elevation of nightly growth hormone release to slightly higher than normal levels results in a major decline in body fat content. This is accomplished by oral ingestion of 500 milligrams of acetyl-l-carnitine and 35-50 milligrams of L-ornithine within an hour of nightly sleep. A obligatory proceeding 3 to 4 hours must have elapsed from the last meal. The high end of this level of intake should not be maintained for more than a few weeks due to the higher growth hormone exposures. GH action to elevate serum free fatty acid for fuel use mostly precludes hunger. GH action to promote anabolic conditions results in little protein loss during this fat reduction.

3.) Elevation of Growth Hormone Release in Cases of Surgery, Trauma, or Catabolic Wasting Disease States.

This application is accomplished by ingestion of an oral mixture of 500 milligrams of acetyl-l-carnitine and 25 to 100 milligrams of L-ornithine. This mixture is also ingested within one hour of nightly sleep at 3 to 4 hours after the last meal. Due to the higher growth hormone secretory effects of this mixture and the variable level of catabolic status, this is best administered each day in a gradual increased level of L-ornithine from the 25 milligrams base value to prevent over stimulation. The cessation of a catabolic state can be monitored by measures of blood urea nitrogen to observe return to normal anabolic to catabolic balance.

4.) Extreme Emergency Awakeness and Alertness Use of High Growth Hormone Augmentation

Oral ingestion of 500 milligrams of acetyl-l-carnitine and 50 to 100 milligrams of l-ornithine within an hour of nightly sleep and at least 3 to 4 hours after the last meal are the conditions for this effect. This administration will almost extinguish the perceived need for sleep. Alertness, mental concentration, and physical strength remain high for a 24 hour period. Due to the very high growth hormone release, this use should only be for a day or two.

5.) Rapid Growth Rates of Immature Domestic Animals by Elevation of Natural (Endogenous) Night time Growth Hormone Release

Abnormally rapid weight gain in immature domesticated animals is accomplished by elevated GH release. This technique scales the intake of the mixture of acetyl-l-carnitine and l-ornithine to the weight of the animal. Oral ingestion takes place at least 3 to 4 hours after the last meal and within an hour of night time sleep. Acetyl-l-carnitine and l-ornithine and are ingested in a 20 to 1 to a 5 to 1 range of ratios. The milligram value of acetyl-l-carnitine is the product of multiplying 8 milligrams by the numerical value of the animal weight in kilograms. The l-ornithine dose is a range of 1 to 4 milligrams multiplied by the numerical weight of the animal in kilograms. This greatly augmented growth hormone release is appropriate to rapid growth of immature domestic animals for subsequent consumption.

Additional/Alternative Embodiment and Operation

Any hypothalamic triggered growth hormone release may also be augmented by this process. This more inclusive embodiment applies to humans and other vertebrates. In addition to night time release, extremely vigorous exercise and normal ovary produced female hormone pulses also trigger GH release. All of these hypothalamic triggered GH releases can also be augmented by prior oral ingestion of the preferred acetyl-l-carnitine and l-ornithine. The preferred choice for the component 1 is acetyl-l-carnitine alone. The preferred choice for the component 2 is l-ornithine alone. Similar pharmacological appropriate dosages apply to the alternative embodiment as they do to the preferred embodiment. As with night time augmentation, this alternative also requires 3 to 4 hour to have elapsed from the last meal. Specifically, no other amino acid intakes are permitted during this fast. Exercise and female hormone triggered GH release magnitudes are quite variable. Reliability of night time release magnitude allows precisely controlled day to day augmented GH release magnitudes. The advantage of this alternative embodiment lies in extending the technique to cover the entire 24 hour period.

Advantages

From the discussion above, a number of advantages of this oral dietary supplement augmentation of GH release are evident:

1.) A precisely controlled augmentation of normal GH release can be accomplished by ingestion of two simple chemical components at a precise temporal relationship to hypothalamic induced GH release.

2.) A very inexpensive oral dietary supplement method can be used to profoundly increase the release of a physiologically important hormonal level.

3.) An unexpected and heretofore unknown synergy between small quantities of these two chemical components allows controlled augmentation of GH release over at least one order of magnitude (one power of 10×), which is far greater than any other simple augmentation technique.

4.) This technique augments normal control mechanisms without inducing untoward feedback consequences.

5.) This technique appears to also augment other hypothalamic triggered hormone releases, and may have multiple beneficial physiological consequences not yet studied.

6.) This technique of augmented GH release with the corresponding secondary physiological benefits may substantially increase the life spans of vertebrates, including humans.

Availability, Purity, and Safety of the Component 1 and the Component 2 Chemicals

Suppliers of high purity acetyl-l-carnitine and L-ornithine are able to document the purity by analysis sheets of which two such documentation sheets (photocopies) will be included in the Information Disclosure Statement section. These compounds are already sold separately as ‘over the counter’ nutritional or dietary supplements. The analysis of purity sheets are included only to establish available commercial sources and purity, and are not the sole or necessarily preferred chemical source supplier for this method.

Both acetyl-l-carnitine and ornithine are naturally occurring compounds normally produced by all mammals, including humans. Their intrinsic safety is indicated by normal biological human serum levels of some 10 micro-molar for acetyl-l-carnitine and 50-60 micro molar levels for l-ornithine. Stored muscle tissue levels of acetyl-l-carnitine or total l-carnitine are orders of magnitude above this treatment level. Acetyl-l-carnitine has undergone long human testing at 2.5-7 grams per day as a successful pharmacological delaying treatment for Alzheimer's disease (19, 20, 21), without reported negative consequences. Oral ingestion of 500 to 2000 milligrams is the maximal immediately absorbed dose for humans (22), but toxicity to this compound is comparable to normal nutritional amino acids that sets in at about 1% of body weight. L-ornithine has been safely administered orally post-surgery at 10 grams/day doses to cause a more rapid return to a positive nitrogen balance than controls (23).

Measurement Evidence of Efficacy

Measurements of GH release occurring at 1.5 hours post sleep were performed on serum samples from a healthy 52 year old male volunteer. Controls and serum analysis for GH were measured by standard commercially avialable antibody capture technique. Experiments and measurement of results occurred between June and October 2000. Each single night experimental serum collection was separated from any other experiments by 1 week. All determinations represent averaged value of triple determinations. Acetyl-l-carnitine and l-ornithine values are in milligrams (mg). Measurements were each performed on serum collected 1.5 hours post night time sleep. Choice of 1.5 hours post sleep represents one complete sleep cycle with slow wave sleep during which GH is released. Oral ingestions of stated substances occurred just before night sleep. The normal range of serum GH at 1.5 hours post sleep is in the 1-10 nanograms per milliliter (ng/ml) range. The variance in measurement precision is given by the second value. All experiments were preceded by a period of 3-4 hours since the last meal.

| | Measured |
|---|---|
| | GH |
| Experimental Conditions | (ng/ml) |
| Control (no treatment) 1.5 hour post sleep serum collection | 0.55 ± 0.2 |
| 500 mg Acetyl-l-carnitine at sleep, 1.5 hour post sleep serum | 0.55 ± 0.1 |
| collection | |
| 500 mg l-ornithine at sleep, 1.5 hour post sleep serum | 0.55 ± 0.2 |
| collection | |
| 500 mg acetyl-l-carnitine + 25 mg l-ornithine at sleep, | 1.25 ± 0.3 |
| 1.5 hour post sleep serum collection | |
| 500 mg acetyl-l-carnitine + 35 mg l-ornithine at sleep, | 7.5 ± 1.2 |
| 1.5 hour post sleep serum collection | |
| 500 mg acetyl-l-carnitine + 45 mg l-ornithine at sleep, | 34.5 ± 3.7 |
| 1.5 hour post sleep serum collection | |

This data shows the absence of effect of either acetyl-l-carnitine or l-ornithine alone to augment GH release at 1.5 hours post sleep. Ingestion of both acetyl-l-carnitine and l-ornithine at night sleep displays a synergy that increases GH release with the level of l-ornithine. This data also indicates a non-linear increase in GH release with increasing l-ornithine in the the active mixture of acetyl-l-carnitine and l-ornithine. This data accords with our choice of 20 to 40 milligrams of l-ornithine along with the 500 milligrams of acetyl-l-carnitine as a proper range for maintenance of young adult levels of augmented human GH release.

Theory of Operation

Both the main and alternative embodiment of this invention appear to work by altering the relative strength of two opposed hypothalamic processes. These hypothalamic processes are somatostatin release and GHRH release. Somatostatin release inhibits growth hormone release by decreasing the level of GHRH release. GHRH release directly promotes growth hormone (GH) release.

Higher blood levels of L-ornithine or L-arginine diminish somatostatin release, possibly by polyamine production. Polyamines are important cellular activation molecules formed biochemically by direct biochemical conversion from L-ornithine. Polyamines act counter to the activation suppression of somatostatin. The eucaryotic polyamine spermine has significant toxicity when administered systematically, but the polyamines putrescine and spermidine are considerably less toxic (24). The fore mentioned GH release by the ‘intravenous arginine provocative test’ elevation of blood levels of the amino acid L-arginine may be taking place by a normal precursor product relationship to also elevate blood L-ornithine levels (25) and thereby polyamines.

Higher blood levels of acetyl-l-carnitine are known to increase mitochondrial energy production capabilities (14). Improved cellular energy production is believed to enable a higher release of GHRH by augmenting cellular energy status. Acety-l-carnitine has well established properties of elevating various neuroactive substances in the brain (16).

The most reliable release of growth hormone occurs in the first 3 hours of night sleep. This has dictated choice of the ‘just at sleep’ night time period for administration of our main embodiment. Administration at any other period of the day has little or no effect, with two exceptions. Administration just before extremely vigorous exercise in either gender augments growth hormone release. Pulsatile release of female estrogens in premenopausal women also augments growth hormone release. Employing this method before the relatively unscheduled estrogen pulses will also augment growth hormone release. Neither exception is as dependable as the night time administration. It is possible to employ the pre-exercise administration at any time of the day. For this reason, this alternative method can be employed for the whole period of 24 hours.

The synergy arising from ingestion of these two components at the same time may arise from these considerations. While the synergy is firmly established by measurement, I have not yet confirmed the underlying theory. Neither compound alone at oral intake levels of 1 gram produce this result.

The mode of action of this synergy also improves the functional status of the hypothalamus in general. This is observed in elderly males that experience a return to more youthful levels of male hormone levels and renewed sexual interest. They also experience a return of testicle size to larger more youthful volume that parallels increases in testosterone levels. This male hormonal enhancement is on a different hormonal axis than GH, but is also a hypothalamic consequence of this method. A generalized enhanced hypothalamic function appears to be the outcome of this chemical synergy.

Conclusion, Ramifications, and Scope

The reader will see that this novel technique offers a multitude of important applications to humans and other vertebrates. This simple oral dietary supplement can reverse the maintenance declines of age that are due to reduced GH levels. Other applications of enormous economic importance include a rapid muscle mass growth of immature domestic animals for the food industry.

While my description contains many specificities, these should not be construed as limitations on the scope of the invention, but rather as exemplification of one preferred embodiment thereof. For example, the not fully specified enhancement of the function of the hypothalamus may entail numerous additional benefits not yet clearly recognized. Another example, is the potential of this technique to massively increase the healthy human life span. We will require many years, probably decades, of both animal and human experience to determine how large this potential enhancement may be. Another example, is the wide variety of secondary consequences of elevated GH levels on body subsystems like: the energy production system, the immune system, the neurological system, the general anabolic conditioning of the body, and improvement in the circadian rhythm entraining system. A very large number of beneficial secondary consequences of this augmented GH release and augmented hypothalamic function is possible, if not probable.

Sequence Listing

(Not Applicable)

1. An oral dietary supplement acting by synergy between two bioactive component substances called a component 1 and a component 2.

2. Said component 1 may be a substance comprising:
a. at least one selected from the group consisting of acetyl-l-carnitine, any acylated ester of l-carnitine having an acyl chain of two to six carbon length, and pharmacological acceptable salts and derivatives thereof and mixtures thereof, and
b. a pharmacological appropriate dose over the range of 10 milligrams to 20 grams.

3. Said component 2 may be a substance comprising:
a. at least one selected from the group consisting of l-ornithine, l-arginine, l-lysine, l-histidine, l-phenylalanine, l-leucine, l-valine, l-methionine, l-threonine, putrescine, spermidine, and pharmacological acceptable salts and derivatives thereof and mixtures thereof, and
b. a pharmacological appropriate dose over the range of 1 milligram to 10 grams.

4. Various pharmacological dosages of the component 1 and the component 2 may be administered by techniques comprising:
a. any appropriate physiological formulation including both solid and liquid formulations and mixtures thereof, and
b. any physiologically appropriate method of delivery of an oral dietary supplement, and
c. separate oral ingestion of the component 1 and the component 2 at approximately the same time, and
d. oral ingestion of a mixture of the component 1 and the component 2 as a single formulation.

5. Ingestion of the component 1 and the component 2 must be preceded by a fast of approximately 3 to 4 hours.

6. A method for augmenting the release of growth hormone in humans by the ingestion of the component 1 and the component 2 for the treatment of conditions and disorders selected from the group consisting of aging decline in GH release, obesity, insufficient GH release in the case of pathology and surgery, emergency needs for prolonged awakeness and physical strength, augmenting the function of the hypothalamus, augmenting the energy production system, augmenting the immune system, augmenting the neurological system, augmenting the general anabolic conditioning of the body, improvement in the circadian rhythm entraining system, exercise related GH release, and premenopausal estrogen spike driven GH release in women.

7. The method claim 6, wherein appropriate pharmacological dose of the component 1 is 500 milligrams and the component 2 dose is 20 to 50 milligrams administered within 1 hour of night time sleep.

8. The method in claim 6, wherein appropriate pharmacological dose of the component 1 is 500 milligrams and the component 2 dose is 20 to 50 milligrams administered 1 hour before extremely vigorous exercise and 1 hour before the large pulastile estrogen release of premenopausal women.

9. A method for augmenting the growth of immature domestic animals by oral ingestion administration of the component 1 and the component 2 within one hour of night time sleep.

10. The method claim 8, wherein the appropriate pharmacological dose of the component 1 is the product of multiplying 8 milligrams by the numerical value of the animal weight in kilograms and the component 2 dose is a range of 1 to 4 milligrams multiplied by the numerical weight of the animal in kilograms.
